(19) United States
(12) Patent Application Publication
(10) Pub. No.: US 2024/0350549 A1
(43) Pub. Date: Oct. 24, 2024

(54) Use of Mesenchymal Stem Cells in Treatment of Juvenile Hypoplastic Left Heart Syndrome

(71) Applicant: Longeveron Inc., Miami, FL (US)

(72) Inventors: Joshua M. HARE, Miami, FL (US); Sunjay KAUSHAL, Miami, FL (US)

(73) Assignee: Longeveron Inc., Miami, FL (US)

(21) Appl. No.: 18/292,150

(22) PCT Filed: Jul. 26, 2022
(86) PCT No.: PCT/US2022/038370

§ 371 Date: Jan. 25, 2024

Related U.S. Application Data
(60) Provisional application No. 63/203,519, filed on Jul. 26, 2021.

(51) Int. Cl. A61K 35/28; A61B 5/00; A61B 5/029; A61P 9/00

(52) U.S. Cl. CPC A61K 35/28; A61B 5/029; A61B 5/4839; A61P 9/00

(57) ABSTRACT

The present disclosure provides methods for treating hypoplastic left heart syndrome in patients in need thereof, the methods involving the administration of a therapeutically effective amount of mesenchymal stem cells. The methods may further involve measuring various biomarkers related to cardiac health and function after administration of the mesenchymal stem cells to determine both the efficacy of the treatment and whether more mesenchymal stem cells need to be administered for a therapeutic effect to occur.

### CROSS-REFERENCE TO RELATED APPLICATIONS

This application is a National Stage Application of International Application No. PCT/US2022/038370 filed on Jul. 26, 2022, which claims the benefit of U.S. Provisional Patent Application No. 63/203,519 filed on Jul. 26, 2021, the contents of which are incorporated herein by reference.

### FIELD OF THE INVENTION

The present disclosure relates to the use of a composition of mesenchymal stem cells in the treatment of juvenile hypoplastic left heart syndrome (HLHS).

### BACKGROUND

Hypoplastic left heart syndrome (HLHS) is a rare cardiac birth defect in which the components of the left ventricle (LV) are variably underdeveloped to the extent that the LV is unable to support systemic circulation (Ohye, R. G. et al. “Comparison of shunt types in the Norwood procedure for single-ventricle lesions”. New England Journal of Medicine, (2010) 362 (21), 1980-1992). The only reason HLHS patients are alive is due to the presence of patent ductus arteriosus (PDA) between the pulmonary artery (PA) and aorta in neonates, which allows the right ventricle (RV) to support systemic circulation. However, the duct naturally closes in the first few days after birth, and in the absence of this duct-dependent systemic circulation, HLHS babies do not survive without early surgical intervention (Barron et al., “Hypoplastic left heart syndrome”. The Lancet, (2009) 374 (9689), 551-564). In addition to an underdeveloped LV, HLHS manifests with variable anatomical defects, including a hypoplastic aorta and aortic arch, and mitral valve atresia or stenosis. Depending on the degree of these abnormalities, HLHS can present with a spectrum of various severities.

In an HLHS heart, deoxygenated blood returns to the right atrium (RA), similar to blood flow seen in a normal heart. But oxygenated blood coming from pulmonary veins into the left atrium (LA), instead of being ejected in LV, traverses into the RA via a defective atrial septum (a patent foramen ovale) and mixes with deoxygenated blood, creating a cyanotic condition. This mixed blood in the RV then proceeds into the PA and splits into two directions. A fraction of this mixed blood flows into the lungs for oxygenation, similar to blood flow seen in a normal heart. The remaining blood flow proceeds into the aorta through a PDA, which enables systemic circulation. However, without intervention, the duct closes and the right side of the heart is no longer be able to support circulation, revealing the insufficiency of the left heart in supporting systemic circulation which has inescapable fatal consequences (Barron et al., 2009; Ohye et al., 2010).

Currently, diagnosis of HLHS is made prenatally in most cases by simply observing the absence of the normal ‘four-chamber’ heart using echocardiography imaging. Although there have been chromosomal and genetic abnormalities associated with HLHS, the genetic factors are variable and heterogeneous (Rychik, J. “Hypoplastic left heart syndrome: from in-utero diagnosis to school age”. Paper presented at the Seminars in Fetal and Neonatal Medicine (2005)).

Although HLHS babies are born with normal body weight and height, growth challenges become apparent with the manifestations of the syndrome after birth and the significant metabolic stress from the necessary open-heart reconstructive surgeries (Kelleher, Laussen, Teixeira-Pinto, & Duggan. “Growth and correlates of nutritional status among infants with hypoplastic left heart syndrome (HLHS) after stage 1 Norwood procedure”. Nutrition, (2006) 22 (3), 237-244). Somatic growth is measured in terms of age- and gender-adjusted Z-scores which is the standard deviation above or below the mean of the general population. A Z-score of 0 is equivalent of 50th percentile, with positive addition going to higher percentiles and vice versa. Kelleher et al. showed that at the time of hospital admission for Stage II operation ˜60% of infants with HLHS were below the fifth weight-for-age percentile (weight-for-age Z score of <−1.65), while ˜40% were below the fifth length-for-age percentile (height-for-age Z score<−1.65). Longer length of hospital stay, longer ICU stay, and frequency of readmissions were independently correlated with poor somatic growth (Kelleher et al., 2006).

As described above, the variably underdeveloped components of LV pose a life-threatening condition in HLHS patients. HLHS is fatal shortly after birth in the absence of surgical intervention, and it accounts for 25% to 40% of all neonatal cardiac mortality (Barron et al., 2009).

The inherent cyanotic nature of HLHS, along with the underdeveloped aorta, also lead to coronary insufficiency, which is a major cause of adverse cardiac events. Additionally, the univentricular status of HLHS even after reconstructive surgery causes abnormal loading conditions in the RV, because the RV serves as the sole systemic pumping chamber. This in turn can trigger detrimental remodeling, despite available cardiac management. Potential manifestations are dilatation (enlargement of the cardiac chamber), myocardial hypertrophy (thickening of the heart walls), and fibrosis (death of cardiac cells which are replaced by scar tissue), which can ultimately lead to heart failure (Wehman et al., “Mesenchymal stem cells preserve neonatal right ventricular function in a porcine model of pressure overload”. Am J Physiol Heart Circ Physiol, (2016) 310 (11), H1816-1826. doi:10.1152/ajpheart.00955.2015). Heart failure can lead to need for heart transplant and/or death.

Management options for HLHS include reconstructive surgery, heart transplantation, and comfort care (also known as compassionate care). These options are time sensitive and parents of HLHS babies undergo a great deal of stress at the time of decision-making (Toebbe, Yehle, Kirkpatrick, & Coddington, “Hypoplastic left heart syndrome: parent support for early decision making”. Journal of pediatric nursing, (2013) 28 (4), 383-392).

The 1-year survival for HLHS babies undergoing reconstructive surgery ranges from 20% to 60% (Siffel, Riehle-Colarusso, Oster, & Correa, “Survival of Children With Hypoplastic Left Heart Syndrome”. Pediatrics, (2015) 136 (4), e864-870. doi:10.1542/peds.2014-1427), and these procedures require several follow-up admissions and additional surgical interventions. Survivors will have limited physical capacity, increased risk of cognitive impairment, and other long term complications (Kon, Ackerson, & Lo, “How pediatricians counsel parents when no best-choice management exists: lessons to be learned from hypoplastic left heart syndrome”. Archives of pediatrics & adolescent medicine, (2004) 158 (5), 436-441). In those cases that opt for reconstructive surgeries, if the clinical outcomes are not favorable post-surgery, enlisting for cardiac transplant is the final end of life option. Regardless, the overall 1-year survival for those undergoing surgery or transplant is ˜40% (Kon et al., 2004), a significant and devastating mortality rate, which calls for novel therapeutic strategies to improve outcomes.

With technical advances in reconstructive surgeries, the survival following each staged procedure has improved over the past decades. However, there is still significant operative mortality, especially with Stage I (Norwood) and the period between Stage I and II (Siffel et al., 2015). Morris et al. reported 26% neonatal mortality (by day 28 of life) in 463 infants with HLHS from a 1999-2007 Texas Birth Defects Registry (Morris et al., “Prenatal diagnosis, birth location, surgical center, and neonatal mortality in infants with hypoplastic left heart syndrome”. Circulation, (2014) 129 (3), 285-292). In-hospital mortality following Norwood surgery was shown to reduce from 40.4% in 1984-1988 era to 15.7% in 2009-2014 (Mascio et al., “Thirty years and 1663 consecutive Norwood procedures: has survival plateaued?” J Thorac Cardiovasc Surg, (2019) 158 (1), 220-229). The one-year survival estimates for HLHS range from 20% up to 74% (Ohye et al., 2010; Siffel et al., 2015). A 2018 study showed that regardless of prenatal vs postnatal diagnosis of HLHS, the 1-year survival is approximately 60% (Alabdulgader, “Survival analysis: prenatal vs. postnatal diagnosis of HLHS”. J Invasive Noninvasive Cardiol, (2018) 1, 8-12). Consistently, Son et al. also demonstrated freedom from death or transplant to be just under 60% at 1-year post-Norwood operation (Son et al., “Prognostic value of serial echocardiography in hypoplastic left heart syndrome”. Circulation: Cardiovascular Imaging, (2018) 11 (7), e006983). In the SVR trial, 6-year transplant-free survival was reported as 60%. So, while we have seen improvements in outcomes, the mortality rate for HLHS patients remains dismal.

Taken together, neonates, infant and children shoulder the heavy burden of morbidity and mortality from HLHS. Even with the most advanced standard of care options, there is significant mortality in the young ages that reaches 60% by 15 years of age (Mahle, Spray, Wernovsky, Gaynor, & Clark III, “Survival after reconstructive surgery for hypoplastic left heart syndrome: a 15-year experience from a single institution”. Circulation, (2000) 102 (suppl_3), lii-136-lii-141). Therefore, novel therapeutic options to increase transplant-free survival and quality of life are desperately needed to improve the current outlook and long-term outcomes of HLHS.

### SUMMARY

The following disclosure contains methods of treatment for HLHS, the methods comprising administering a composition of mesenchymal stem cells (MSC) to a subject in need of HLHS treatment.

### BRIEF DESCRIPTION OF THE DRAWINGS

FIG. 1 depicts right ventricular mass changes for each patient throughout the course of a clinical study. The data was indexed according to the body surface area (BSA) of the patients.

FIG. 2 depicts right ventricular ejection fraction changes for each patient throughout the course of a clinical study.

FIG. 3 depicts right ventricular end-systolic volume changes for each patient throughout the course of a clinical study. The data was indexed to the BSA of the patients.

FIG. 4 depicts right ventricular end-diastolic volume changes for each patient throughout the course of a clinical study. The data was indexed to the BSA of the patients.

FIG. 5 depicts stroke volume changes for each patient throughout the course of a clinical study. The data was indexed to the BSA of the patients.

FIG. 6 depicts the change in the length-for-age Z-scores of each patient throughout the course of a clinical study.

FIG. 7 depicts the change in the weight-for-age Z-scores of each patient throughout the course of a clinical study.

FIG. 8 depicts the change in systolic blood pressure for each patient throughout the course of a clinical study.

FIG. 9 depicts the change in diastolic blood pressure for each patient throughout the course of a clinical study.

FIG. 10 depicts the change in heart rate for each patient throughout the course of a clinical study.

FIG. 11 depicts the change in tricuspid regurgitation fraction for select patients throughout the course of a clinical study.

FIG. 12 depicts the change in tricuspid regurgitation net aortic forward flow for select patients throughout the course of a clinical study.

FIG. 13 depicts the change in tricuspid regurgitation for each patient throughout the course of a clinical study.

FIG. 14 depicts a comparison between the post-treatment survival rate of patients who were administered Lomecel-B™ cells for treatment of HLHS and patients who underwent the clinical study performed by Son, et al. for treatment of HLHS.

### DETAILED DESCRIPTION

MSCs are multipotent cells that are immunoprivileged and able to migrate to sites of injury and inflammation (Klyushnenkova et al., “Growth and correlates of nutritional status among infants with hypoplastic left heart syndrome (HLHS) after stage 1 Norwood procedure”. Nutrition, (2006) 22 (3), 237-244; Le Blanc et al., “Mesenchymal stem cells for treatment of steroid-resistant, severe, acute graft-versus-host disease: a phase II study”. Lancet, (2008) 371 (9624), 1579-1586. doi: 10.1016/S0140-6736 (08) 60690-X). The exact mechanism of action of MSCs is yet to be fully elucidated, but it appears to involve a complex orchestration with host cells (Hatzistergos et al., “Bone marrow mesenchymal stem cells stimulate cardiac stem cell proliferation and differentiation”. Circ Res, (2010) 107 (7), 913-922; A. R. Williams et al., “Enhanced effect of combining human cardiac stem cells and bone marrow mesenchymal stem cells to reduce infarct size and to restore cardiac function after myocardial infarction”. Circulation, (2013) 127 (2), 213-223. doi: 10.1161/CIRCULATIONAHA. 112.131110 2013; A. R. Williams et al., “Intramyocardial stem cell injection in patients with ischemic cardiomyopathy: functional recovery and reverse remodeling”. Circ Res, (2011) 108 (7), 792-796. doi: 10.1161/CIRCRESAHA. 111.242610). MSCs have demonstrated a potential for clinical benefit in cardiovascular disease via their pro-angiogenic and anti-inflammatory properties (Cao et al., “S-nitrosoglutathione reductase-dependent PPARgamma denitrosylation participates in MSC-derived adipogenesis and osteogenesis”. J Clin Invest, (2015) 125 (4), 1679-1691. doi: 10.1172/jci73780; Hatzistergos et al.; A. R. Williams & Hare, J. M., “Mesenchymal stem cells: biology, pathophysiology, translational findings, and therapeutic implications for cardiac disease”. Circ Res, (2011) 109 (8), 923-940. doi: 10.1161/CIRCRESAHA. 111.243147).

MSCs secrete numerous bioactive molecules that: stimulate endogenous stem cell recruitment, proliferation, and differentiation; inhibit apoptosis and fibrosis; and stimulate neovascularization. MSCs can also regulate host stem cell niches through cell-cell interactions. Thus, MSCs can enhance intrinsic repair and regenerative mechanisms. Preclinical studies have shown that MSCs promote cardiac repair/regeneration directly through formation of new tissue, and indirectly through paracrine effects (Malliaras, Kreke, & Marban, “The stuttering progress of cell therapy for heart disease”. Clin Pharmacol Ther, (2011) 90 (4), 532-541. doi: 10.1038/clpt.2011.175; Rosen, Myerburg, Francis, Cole, & Marban, “Translating stem cell research to cardiac disease therapies: pitfalls and prospects for improvement”. J Am Coll Cardiol, (2014) 64 (9), 922-937. doi: 10.1016/j.jacc.2014.06.1175).

Accordingly, we have surprisingly discovered that the use of a composition comprising MSCs is able to combat the symptoms of HLHS. Treating a patient suffering from HLHS symptoms with a composition comprising MSCs has been discovered to improve the subject's cardiac morphology and function. The above discoveries are surprising due to the general reservation of those skilled in the art to use MSCs in treatments for HLHS since they were expected to perform poorly due to their low residence time in the human body.

Following the surprising discoveries above, one objective of the present disclosure is to provide methods of treatment or alleviation for HLHS that comprise administering a therapeutic amount of MSCs to a subject in need thereof to alleviate the symptoms and/or treat the progression of HLHS. The efficacy of the treatment methods disclosed herein can be determined by measuring the changes in biomarkers related to cardiac health and function. These biomarkers can be the change in the patient's right ventricular mass, right ventricular ejection fraction, right ventricular end-systolic volume, right ventricular end-diastolic volume, stroke volume, length-for-age Z-scores, weight-for-age Z-scores, systolic blood pressure, diastolic blood pressure, heart rate or any combination thereof after administration and/or treatment with MSCs. Accordingly, the treatment methods disclosed herein can comprise measuring any of the above biomarkers before and/or after administration of MSCs to the patient. These biomarkers can be measured to determine the efficacy of the treatment and whether more mesenchymal stem cells need to be administered for a therapeutic effect to occur.

As used herein, the term “therapeutic effect” includes, but is not limited to, any improvement in the patient's cardiac function or health after administration of the MSCs.

As used herein, the term “patient” includes, but is not limited to, humans and non-human vertebrates such as wild, domestic, and farm animals. In some embodiments, the term refers to juvenile humans <18 years of age. In some embodiments, the human patient exhibits symptoms of HLHS.

In some embodiments, the treatment methods comprise measuring the change in the patient's right ventricular mass after administration of MSCs. In exemplary embodiments, the patients right ventricular mass is increased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's right ventricular mass after administration of MSCs is increased to a stable mass wherein the mass does not decline more than 0.1% to 10%, 0.1% to 5% or 0.1% to 1% once it has reached and maintained a mass that is different from the mass before administration of MSCs to the patient in need thereof.

In other embodiments, the treatment methods comprise measuring the change in the patient's right ventricular ejection fraction after administration of MSCs. In exemplary embodiments, the patients right ventricular ejection fraction is decreased after administration of MSCs in the range from 0.1% to 10%, 0.5% to 10%, 1.0% to 10%, 3% to 10%, 1% to 5%, 1% to 3%, greater than 0% to less than or equal to 5%, 10% to 50%, 20% to 50%, 30% to 50% or greater than 50%. In other exemplary embodiments, the change in the patient's right ventricular ejection fraction after administration of MSCs is decreased to a stable level wherein the right ventricular ejection fraction does not increase more than 0.1% to 10%, 0.1% to 5% or 0.1% to 1% once it has reached and maintained an ejection fraction that is different from the ejection fraction before administration of MSCs to the patient in need thereof.

In some embodiments, the treatment methods comprise measuring the change in the patient's right ventricular end-systolic volume after administration of MSCs. In exemplary embodiments, the patients right ventricular end-systolic volume is increased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's right ventricular end-systolic volume after administration of MSCs is increased to a stable volume wherein the volume does not decline more than 0.1% to 10%, 0.1% to 5% or 0.1% to 1% once it has reached and maintained a volume that is different from the volume before administration of MSCs to the patient in need thereof.

In other embodiments, the treatment methods comprise measuring the change in the patient's right ventricular end-diastolic volume after administration of MSCs. In exemplary embodiments, the patients right ventricular end-diastolic volume is increased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's right ventricular end-diastolic volume after administration of MSCs is increased to a stable volume wherein the mass does not decline more than 0.1% to 10%, 0.1% to 5% or 0.1% to 1% once it has reached and maintained a volume that is different from the volume before administration of MSCs to the patient in need thereof.

In some embodiments, the treatment methods comprise measuring the change in the patient's stroke volume after administration of MSCs. In exemplary embodiments, the patients stroke volume is decreased after administration of MSCs in the range from 0.1% to 10%, 0.5% to 10%, 1.0% to 10%, 3% to 10%, 1% to 5%, 1% to 3%, greater than 0% to less than or equal to 5%, 10% to 50%, 20% to 50%, 30% to 50% or greater than 50%. In other exemplary embodiments, the change in the patient's stroke volume after administration of MSCs is decreased to a stable level wherein the stroke volume does not increase more than 0.1% to 10%, 0.1% to 5% or 0.1% to 1% once it has reached and maintained a volume that is different from the volume before administration of MSCs to the patient in need thereof.

In some embodiments, the treatment methods comprise measuring the change in the patient's length-for-age Z-score after administration of MSCs. In exemplary embodiments, the patients length-for-age Z-score is increased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's length-for-age Z-score after administration of MSCs is increased to a stable level wherein the Z-score does not decline more than 0.1% to 10%, 0.1% to 5% or 0.1% to 1% once it has reached and maintained a Z-score that is different from the Z-score before administration of MSCs to the patient in need thereof.

In some embodiments, the treatment methods comprise measuring the change in the patient's weight-for-age Z-score after administration of MSCs. In exemplary embodiments, the patients weight-for-age Z-score is increased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's weight-for-age Z-score after administration of MSCs is increased to a stable level wherein the Z-score does not decline more than 0.1% to 10%, 0.1% to 5% or 0.1% to 1% once it has reached and maintained a Z-score that is different from the Z-score before administration of MSCs to the patient in need thereof.

In some embodiments, the treatment methods comprise measuring the change in the patient's systolic blood pressure after administration of MSCs. In exemplary embodiments, the patients systolic blood pressure is increased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's systolic blood pressure after administration of MSCs is increased to a stable pressure wherein the pressure does not decline more than 0.1% to 10%, 0.1% to 5% or 0.1% to 1% once it has reached and maintained a pressure that is different from the pressure before administration of MSCs to the patient in need thereof.

In some embodiments, the treatment methods comprise measuring the change in the patient's diastolic blood pressure after administration of MSCs. In exemplary embodiments, the patients diastolic blood pressure is changed after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's diastolic blood pressure after administration of MSCs is changed to a stable pressure wherein the pressure does not change more than 0.1% to 10%, 0.1% to 5% or 0.1% to 1% once it has reached and maintained a pressure that is different from the pressure before administration of MSCs to the patient in need thereof.

In some embodiments, the treatment methods comprise measuring the change in the patient's heart rate after administration of MSCs. In exemplary embodiments, the patient's heart rate is changed after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's heart rate after administration of MSCs is changed to a stable rate wherein the rate does not change more than 0.1% to 10%, 0.1% to 5% or 0.1% to 1% once it has reached and maintained a rate that is different from the rate before administration of MSCs to the patient in need thereof.

In some embodiments, the treatment methods comprise measuring the change in the patient's tricuspid regurgitation after administration of MSCs. In exemplary embodiments, the patient's tricuspid regurgitation is improved from a severe state to either a moderate or mild state.

In other embodiments, the treatment methods comprise measuring the change in the patient's tricuspid regurgitation fraction after administration of MSCs. In exemplary embodiments, the patient's tricuspid regurgitation fraction is decreased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's tricuspid regurgitation fraction after administration of MSCs is decreased to a stable fraction wherein the fraction does not decline more than 0.1% to 10%, 0.1% to 5% or 0.1% to 1% once it has reached and maintained a fraction that is different from the fraction before administration of MSCs to the patient in need thereof.

In other embodiments, the treatment methods comprise measuring the change in the patient's tricuspid regurgitation net aortic forward flow after administration of MSCs. In exemplary embodiments, the patient's tricuspid regurgitation net aortic forward flow is increased after administration of MSCs in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other exemplary embodiments, the change in the patient's tricuspid regurgitation net aortic forward flow after administration of MSCs is increased to a stable net aortic forward flow wherein the net aortic forward flow does not increase more than 0.1% to 10%, 0.1% to 5% or 0.1% to 1% once it has reached and maintained a net aortic forward flow that is different from the net aortic forward flow before administration of MSCs to the patient in need thereof.

In other embodiments, the treatment methods comprise measuring the survival rate of the patient after administration of MSCs. In exemplary embodiments, the survival rate of the patient increased in the range from 0.1% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50% after administration of MSCs.

The composition of mesenchymal stem cells used in embodiments of the invention can include isolated allogeneic human mesenchymal stem cells derived from either the bone marrow and/or adipose tissue or LOMECEL-B™ cells (Longeveron formulation of allogenic human mesenchymal stem cells) which are reported in the following United States Patent Application Publications, all of which are incorporated by reference herein: US20190038742A1; US20190290698 A1; and US20200129558A1.

As used herein, the term “allogeneic” refers to a cell that is of the same animal species but genetically different in one or more genetic loci as the animal that becomes the “recipient host.” This usually applies to cells transplanted from one animal to another non-identical animal of the same species.

In exemplary embodiments, the MSCs are administered in a therapeutically effective amount of about 1×10⁶, 2×10⁶, 5×10⁶, 10×10⁶, 20×10⁶, 30×10⁶, 40×10⁶, 50×10⁶, 60×10⁶, 70×10⁶, 80×10⁶, 90×10⁶, 100×10⁶, 110×10⁶, 120×10⁶, 130×10⁶, 140×10⁶, 150×10⁶, 160×10⁶, 170×10⁶, 180×10⁶, 190×10⁶, 200×10⁶, 300×10⁶, 400×10⁶, 500×10⁶, 10×10⁷ or any amount between 20×10⁶ and 100×10⁶ MSCs.

As used herein, a “therapeutically effective amount” means an amount of MSCs that stimulates an improvement in cardiac function. Such an improvement can be characterized by the heart's ability to grow to higher right ventricular masses or elicit higher end-diastolic/end-systolic volumes. The dosage and number of doses (e.g., single or multiple dose) administered to the patient will vary depending upon a variety of factors, including the route of administration, patient conditions and characteristics (sex, age, body weight, health, size), extent of symptoms, concurrent treatments, frequency of treatment and the effect desired, and the like.

In exemplary embodiments, the patient is between 1 to 15 years old, 3 to 15 years old, 3 to 10 years old, 5 to 10 years old or 5 to 15 years old. In some embodiments the patient is under 1 year old.

In other exemplary embodiments, the treatment methods further comprise measuring the change in the biomarkers disclosed herein directly after administration, one month after administration, two months after administration, six months after administration, nine months after administration or any time from the beginning of administration to 12 months after administration.

In exemplary embodiments, the MSCs are administered as a single dose. In another embodiments, the MSCs are administered in multiple doses, e.g. two or more doses. In other embodiments, the MSCs are administered at least yearly.

In other exemplary embodiments, the administration of the MSCs is repeated, such as at least 1, 2, 3, 4, 5, 6, 7, 8, 9, 10, 11, 12, 13, 14, 15, 16, 17, or 18 months after the first administration of the isolated population of MSCs, or repeated between 2-4, 2-6, 2-8, 2-10, 3-4, 3-6, 3-8, 3-10, 4-6, 4-8, 4-10, 6-8, 6-10, 6-12, or 12-18 months after the first administration of the MSCs.

### EXAMPLES

### Example 1

This example is based on a phase I clinical study involving the use of mesenchymal stem cells to treat juvenile HLHS. This phase I study was an open-label design titled “Longeveron Mesenchymal Stem Cells (LMSCs) Delivered during Stage II Surgery for Hypoplastic Left Heart Syndrome (ELPIS Phase I)”. The objective was to evaluate the safety and feasibility of intramyocardial injection of Lomecel-B™ product into HLHS patients during Stage II reconstructive surgery in 10 consecutive patients who met the enrollment criteria (Kaushal et al., “Study design and rationale for ELPIS: A phase I/IIb randomized pilot study of allogeneic human mesenchymal stem cell injection in patients with hypoplastic left heart syndrome”. American heart journal, (2017) 192, 48-56. doi: https://doi.org/10.1016/j.ahj.2017.06.009).

This study enrolled 10 HLHS patients requiring Stage II surgery. Major exclusion criteria were restrictive or intact atrial septum, presence of significant coronary artery sinusoids, patients requiring mechanical circulatory support prior to surgery, and evidence of arrhythmia requiring anti-arrhythmia therapy. Once the patient was on cardiopulmonary bypass for the Stage II operation, Lomecel-B™ product was delivered at 2.5×10⁶ cells/kg of body weight via intramyocardial injections using a 27-gauge needle syringe at the completion of the repair, but before separating from cardiopulmonary bypass. Baseline assessments were performed prior to Stage II reconstructive operation and follow-ups at 6 and 12 months after surgery were performed to evaluate safety and provisional clinical outcomes including cardiac function by MRI.

The following primary (safety) and secondary (efficacy) endpoints were measured and monitored during the clinical study.

The primary endpoints included:
incidence of major adverse cardiac events through 1-year post-treatment, including:
   sustained/symptomatic ventricular tachycardia requiring intervention with inotropic support;
   aggravation of heart failure;
   myocardial infarction;
   unplanned cardiovascular operation for cardiac tamponade; and
   death; and
infection during the first month post-treatment.

The secondary endpoints included:
a change from baseline in:
   right ventricular function;
   right ventricular end-diastolic volume;
   right ventricular end-systolic volume;
   right ventricular end-systolic diameter;
   tricuspid regurgitation measured by serial echocardiograms and MRI.
change in somatic growth (weight, height, head circumference); and
assessment of co-morbidity, including:
   cardiovascular morbidity;
   need for transplantation;
   re-hospitalizations;
   cardiovascular mortality; and
   all-cause mortality.

### Patient Population

Table 1 summarizes the demographics and baseline characteristics of the study population. Ten patients undergoing Stage II reconstruction were successfully treated with Lomecel-B™ product. The cohort included 7 males and 3 females, all non-Hispanic; 7 were White and 3 were African American, with a mean of 4.89±0.85 months of age at the time of Stage II surgery. All patients successfully underwent the Stage II surgery during which Lomecel-B™ product injections were delivered. Mean length of hospital stay was 11.7±9.58 days. All of the patients had a RV-PA shunt at Stage I (Norwood). Other baseline features, including cardiac parameters measured by MRI, are presented in Table 1.

TABLE 1
Baseline demographics of ELPIS Phase I patients

| Characteristic | N = 10 | |
|---|---|---|
| Age at Glenn Operation (months) | 4.89 ± 0.85 | |
| Male gender [n (%)] | 7 | (70.0) |
| Ethnicity [n (%)] | | |
| Hispanic or Latino | 0 | |
| Not Hispanic or Latino | 10 | (100.0) |
| Race [n (%)] | | |
| White | 7 | (70.0) |
| Black/African American | 3 | (30.0) |
| Body Surface Area (BSA) (m²) | 0.331 ± 0.03 | |
| Length for Age Z-Score | −0.87 ± 2.02 | |
| Weight for Age Z-Score | 1.09 ± 1.31 | |
| Duration of Bypass (min) | 113.1 ± 17.44 | |
| Duration of Injection (min) | 5.5 ± 1.78 | |
| Hospital Length of Stay (days) | 11.7 ± 9.58 | |
| Norwood Shunt Type [n (%)] | | |
| RV-PA (Sano) | 10 | (100) |

| | |
|---|---|
| RV Function by MRI | |
| RV Ejection Fraction (%) | 62.62 ± 5.99 |
| RV End Systolic Volume Index (mL/m² BSA¹³) | 49.71 ± 15.61 |
| RV End Diastolic Volume Index (mL/m² BSA¹³) | 133.59 ± 36.14 |
| RV Stroke Volume Index (mL/m² BSA¹³) | 83.88 ± 23.65 |
| RV Mass Index (g/m² BSA¹³) | 101.26 ± 33.71 |
| TR fraction | 0.55 ± 0.05 |
| Neo-Aortic Forward Flow | 11 ± 2.65 |
| RV systolic diameter (mm/m² ✓BSA) | 37.5 ± 9.24 |
| RV diastolic diameter (mm/m² ✓BSA) | 61.36 ± 13.95 |
| GLS (%) | −24.39 ± 6.99 |
| Sphericity Index | 1.3 ± 0.35 |

The sphericity index of each patient was determined using the following formula: Sphericity=RV Length (D)/(RVDs SAX A/P).

### Safety Findings

Intramyocardial injection of Lomecel-B™ product was well-tolerated, with no MACE, and no infections or any other adverse events reported that were considered to be related to investigational treatment.

### Efficacy Findings

The following data is presented as mean±SD. Data was collected from multiple sites. Statistical analysis was performed using GraphPad Prism v9.2. One-Way ANOVA with Mixed-Effects Model was used for multiple comparisons with Bonferroni correction. An alpha of <0.05 was considered statistically significant.

The BSA of each patient was determined using the Haycock Formula (BSA=0.024265·h^{0.3964}·w^{0.5378}, h=the height of the patient (cm) and w=the weight of the patient (kg)).

The efficacy of the clinical study was evaluated by determining whether there was any significant change in any of the secondary endpoints after administration of Lomecel-B™ cells to the patients. These secondary endpoints were measured through the use of echocardiograms and magnetic resonance imaging (MRI). Table 2 contains the secondary endpoint MRI data for all treatment groups (including the Longeveron study referred to above plus four additional patients), the data being indexed to BSA. Table 3 contains the secondary endpoint MRI data for only the Lomecel-B™ product treatments, the data being indexed to BSA. Each * represents a p<0.05 compared to baseline. Each ** represents a p<0.01 compared to baseline. Each *** represents a p<0.001 compared to baseline.

TABLE 2
Secondary Endpoint MRI Data for All Treatment Groups

| | | RV End | RV End | | |
|---|---|---|---|---|---|
| | | Systolic | Diastolic | RV Stroke | RV mass |
| All treatments | BSA (m2) | Volume (mL) | Volume (mL) | Volume (mL) | (g) |
| Baseline | 0.33 ± 0.029 | 12.36 ± 5.93 | 33.71 ± 11.81 | 21.36 ± [symbol] | 25 ± 6.46 |
| 6-month | 0.427 ± 0.032 | 25 ± 12.05 | 54.5 ± 19.06 | 29.5 ± 9.4 | 38.83 ± 12.84 |
| 12-month | 0.471 ± 0.036 | [symbol] | 56.08 ± 19.73 | 29.25 ± 7.41 | 37.42 ± 10.55 |
| 6-month Change | 0.099 ± 0.028 *** | 12.5 ± 8.05 *** | 20.75 ± 13.18 *** | 8.25 ± 8.44 ** | 13.42 ± 7.43 *** |
| from Baseline | | | | | |
| 12-month Change | 0.139 ± 0.034 *** | 14.67 ± 11.68 *** | 22.33 ± 15.14 *** | 7.67 ± 9.18 * | 13.17 ± 8.41 *** |
| from Baseline | | | | | |

| | | RV End | RV End | | |
|---|---|---|---|---|---|
| | | Systolic | Diastolic | RV Stroke | RV Mass |
| | RV Ejection | Volume Index | Volume Index | Volume Index | Index |
| All treatments | Fraction (%) | (mL/m2) | (mL/m2) | (mL/m2) | (g/m2) |
| Baseline | 64.15 ± 7.69 | 53.04 ± 27.2 | 144.06 ± 53.62 | 91.02 ± 29 | 106.52 ± 33.43 |
| 6-month | 54.86 ± 9.6 | 75.18 ± 33.46 | 165.18 ± [symbol] | 90 ± 30.26 | 116.5 ± 32.02 |
| 12-month | 53.69 ± 9.95 | 72 ± 44.01 | 150.42 ± 55.4 | 78.42 ± 20.71 | 100.39 ± 31.03 |
| 6-month Change | −9.11 ± 9.45 ** | 20.68 ± 21.08 ** | 18.67 ± 41.46 | −2.01 ± [symbol] | 7.75 ± 25.84 |
| from Baseline | | | | | |
| 12-month Change | −11.09 ± 9.77 ** | 19.72 ± 25.25 * | 6.41 ± 44.24 | −13.31 ± 34.21 | −2.1 ± 28.8 |
| from Baseline | | | | | |

[symbol]

indicates data missing or illegible when filed

TABLE 3
Secondary Endpoint MRI Data for Lomecel-B ™ Treatment Groups

| | | RV End | RV End | | |
|---|---|---|---|---|---|
| | | Systolic | Diastolic | RV Stroke | RV mass |
| Lomecel-B-treated | BSA (m2) | Volume (mL) | Volume (mL) | Volume (mL) | (g) |
| Baseline | 0.331 ± 0.03 | 11.8 ± 3.85 | 31.7 ± [symbol] | [symbol] | 24.2 ± [symbol] |
| 6-month | 0.432 ± 0.03 | 23.67 ± 8.54 | 50.89 ± 11.38 | 27.22 ± [symbol] | 37.44 ± 12.41 |
| 12-month | 0.478 ± 0.034 | 23.88 ± 3.68 | 50.5 ± 8.94 | [symbol] | 37 ± [symbol] |
| 6-month Change | 0.105 ± 0.03 *** | 11.67 ± 7.95 *** | 18.67 ± 12.44 ** | 7 ± 8.67 * | 13.11 ± 7.69 *** |
| from Baseline | | | | | |
| 12-month Change | 0.144 ± 0.037 *** | 12.5 ± 4.9 *** | 19.25 ± 11.76 ** | 6.75 ± 9.11 | 14.13 ± 7.1 *** |
| from Baseline | | | | | |

| | | RV End | RV End | | |
|---|---|---|---|---|---|
| | | Systolic | Diastolic | RV Stroke | RV Mass |
| | RV Ejection | Volume Index | Volume Index | Volume Index | Index |
| Lomecel-B-treated | Fraction (%) | (mL/m2) | (mL/m2) | (mL/m2) | (g/m2) |
| Baseline | [symbol] | 49.71 ± 15.61 | 133.59 ± 36.14 | [symbol] ± 23.65 | 101.28 ± 33.71 |
| 6-month | 53.69 ± 9.56 | [symbol] | 152.07 ± 32.09 | 82.22 ± 25.25 | 110.82 ± [symbol] |
| 12-month | 52.31 ± 5.63 | 63.03 ± 13.39 | 133.44 ± 31.42 | 70.41 ± 20.5 | 97.88 ± 24.22 |
| 6-month Change | −8.89 ± 10.93 * | 18.69 ± 24.2 * | [symbol] ± 46.73 | [symbol] ± 33.35 | [symbol] ± 30.22 |
| from Baseline | | | | | |
| 12-month Change | −10.88 ± 10.7 * | 15.3 ± 19.42 | 2.55 ± 48.04 | −12.75 ± 34.8 | 2.23 ± 33.48 |
| from Baseline | | | | | |

[symbol]

indicates data missing or illegible when filed

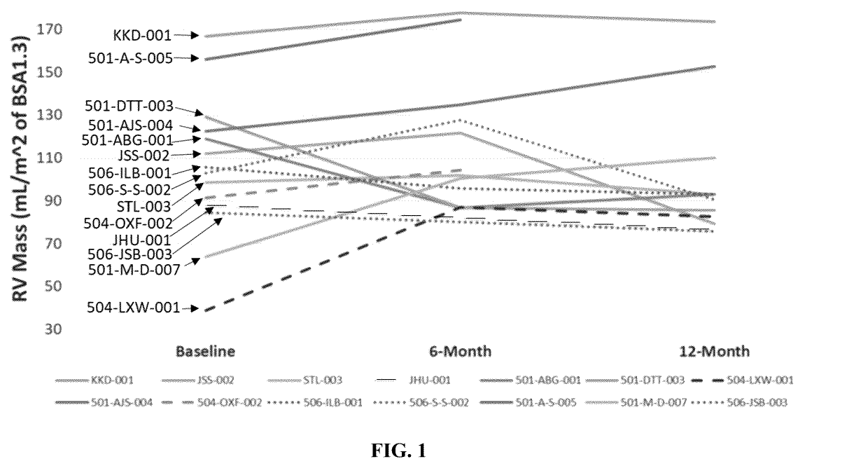

FIG. 1 depicts right ventricular mass changes for each patient throughout the course of the clinical study. Measurements were taken at the beginning of the clinical study, six months post administration and twelve months post administration. The data presented within FIG. 1 was indexed according to the BSA of the patients. Table 4 contains MRI data used to determine the change in each patient's right ventricular mass after administration of the Lomecel-B™ cells.

TABLE 4
Change in the Right Ventricular Mass of Each Patient
after Lomecel-B ™ Administration

| | RV Mass | RV Mass | RV Mass |
|---|---|---|---|
| | Baseline | 6-Month | 12-Month |
| | (mL/m² | (mL/m² | (mL/m² |
| Patient (ID #) | of BSA) | of BSA) | of BSA) |

| | | | | |
|---|---|---|---|---|
| 1 | (KKD-001) | 166.4868 | 177.5162 | 173.4309 |
| 2 | (JSS-002) | 111.8801 | 121.4127 | 79.12428 |
| 3 | (STL-003) | 98.43316 | 101.6927 | 93.01269 |
| 4 | (JHU-001) | 87.81953 | | 76.04896 |
| 5 | (501-ABG-001) | 118.7652 | 86.58227 | 93.02765 |
| 6 | (501-DTT-003) | 128.8587 | 86.71631 | 85.63465 |
| 7 | (504-LXW-001) | 38.72818 | 86.8293 | 82.62783 |
| 8 | (501-AJS-004) | 122.3044 | 134.8654 | 152.5436 |
| 9 | (504-OXF-002) | 91.37495 | 104.3089 | |
| 10 | (506-ILB-001) | 105.5918 | 95.75959 | 93.08321 |
| 11 | (506-S-S-002) | 102.8123 | 127.4662 | 90.44972 |
| 12 | (501-A-S-005) | 156.0051 | 174.4393 | |
| 13 | (501-M-D-007) | 63.75947 | 100.4393 | 109.9048 |
| 14 | (506-JSB-003) | 84.39177 | | 75.76821 |

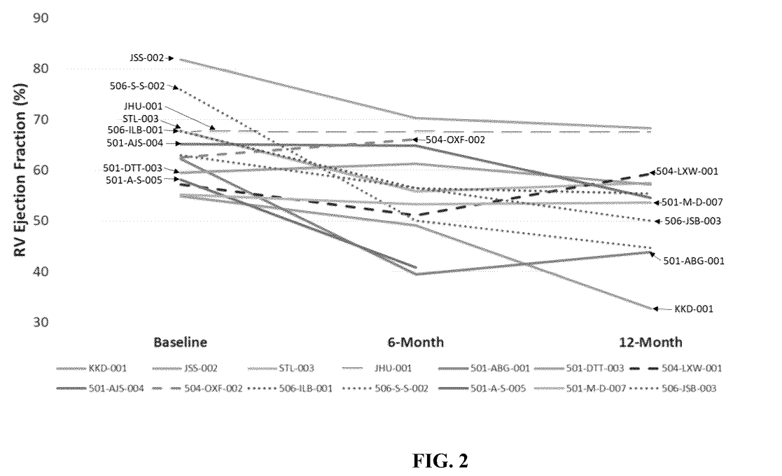

FIG. 2 depicts the right ventricular ejection fraction changes for each patient throughout the course of the clinical study. Measurements were taken at the beginning of the clinical study, six months post administration and twelve months post administration. Table 5 contains the MRI data that was used to determine the change in each patient's right ventricular ejection fraction after administration of the Lomecel-B™ cells.

TABLE 5
Change in the Right Ventricular Ejection Fraction of Each
Patient after Lomecel-B ™ Administration

| | | EF | EF |
|---|---|---|---|
| | EF Baseline | 6-Month | 12-Month |
| Patient (ID #) | (%) | (%) | (%) |

| | | | | |
|---|---|---|---|---|
| 1 | (KKD-001) | 54.83871 | 49.0566 | 32.74336 |
| 2 | (JSS-002) | 81.81818 | 70.21277 | 68.25397 |
| 3 | (STL-003) | 67.74194 | 55.81395 | 57.44681 |
| 4 | (JHU-001) | 67.5 | | 67.3913 |
| 5 | (501-ABG-001) | 62.16216 | 39.47368 | 43.90244 |
| 6 | (501-DTT-003) | 59.45946 | 61.22449 | 57.14286 |
| 7 | (504-LXW-001) | 57.14286 | 51.06383 | 59.25926 |
| 8 | (501-AJS-004) | 65.11628 | 64.81481 | 54.54545 |
| 9 | (504-OXF-002) | 62.5 | 66.07143 | |
| 10 | (506-ILB-001) | 67.64706 | 56.41026 | 55.35714 |
| 11 | (506-S-S-002) | 75.86207 | 50 | 44.68085 |
| 12 | (501-A-S-005) | 58.13953 | 40.78947 | |
| 13 | (501-M-D-007) | 55.17241 | 53.33333 | 53.57143 |
| 14 | (506-JSB-003) | 62.96296 | | 50 |

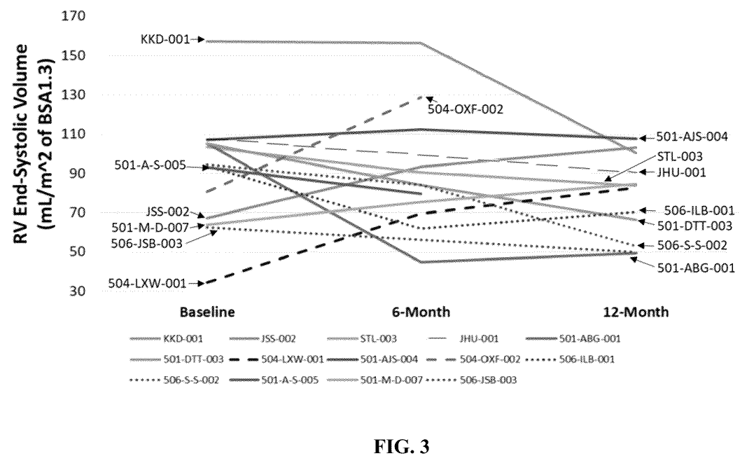

FIG. 3 depicts the right ventricular end-systolic volume changes for each patient throughout the course of the clinical study. Measurements were taken at the beginning of the clinical study, six months post administration and twelve months post administration. The data presented within FIG. 3 was indexed to the BSA of the patients. Table 6 contains the MRI data used to determine the change in each patient's right ventricular end-systolic volume after administration of the Lomecel-B™ cells.

TABLE 6
Change in the Right Ventricular End-Systolic Volume of Each
Patient after Lomecel-B ™ Administration

| | RV ESV | RV ESV | RV ESV |
|---|---|---|---|
| | Baseline | 6-Month | 12-Month |
| | (mL/m² | (mL/m² | (mL/m² |
| Patient (ID #) | of BSA) | of BSA) | of BSA) |

| | | | | |
|---|---|---|---|---|
| 1 | (KKD-001) | 129.4897 | 162.4725 | 205.9492 |
| 2 | (JSS-002) | 14.91734 | 39.52973 | 47.95411 |
| 3 | (STL-003) | 49.21658 | 71.56151 | 62.00846 |
| 4 | (JHU-001) | 51.89336 | | 43.8744 |
| 5 | (501-ABG-001) | 63.9505 | 68.6687 | 62.93047 |
| 6 | (501-DTT-003) | 71.58819 | 53.1487 | 49.72334 |
| 7 | (504-LXW-001) | 25.81879 | 66.56913 | 56.80663 |
| 8 | (501-AJS-004) | 57.33017 | 61.01055 | 89.73151 |
| 9 | (504-OXF-002) | 48.37497 | 66.06233 | |
| 10 | (506-ILB-001) | 44.67345 | 47.8798 | 56.75806 |
| 11 | (506-S-S-002) | 29.98691 | 83.94114 | 65.3248 |
| 12 | (501-A-S-005) | 66.85935 | 115.4378 | |
| 13 | (501-M-D-007) | 51.80457 | 65.91331 | 73.26988 |
| 14 | (506-JSB-003) | 36.69207 | | 49.72289 |

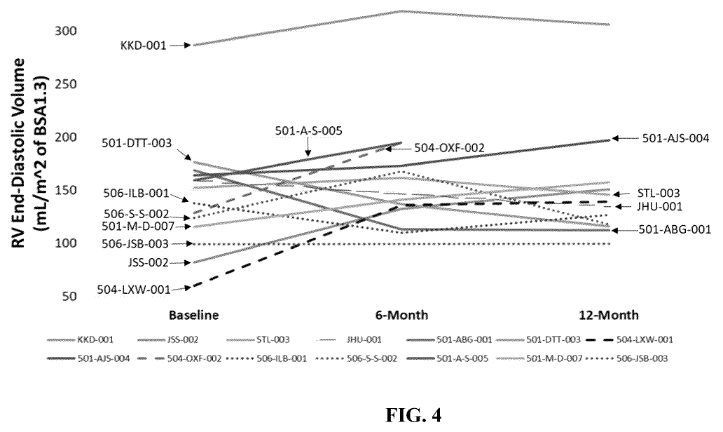

FIG. 4 depicts the right ventricular end-diastolic volume changes for each patient throughout the course of the clinical study. Measurements were taken at the beginning of the clinical study, six months post administration and twelve months post administration. The data presented within FIG. 4 was indexed to the BSA of the patients. Table 7 contains MRI data used to determine the change in each patient's right ventricular end-diastolic volume after administration of the Lomecel-B™ cells.

TABLE 7
Change in the Right Ventricular End-Diastolic Volume of Each
Patient after Lomecel-B ™ Administration

| | RV EDV | RV EDV | RV EDV |
|---|---|---|---|
| | Baseline | 6-Month | 12-Month |
| | (mL/m² | (mL/m² | (mL/m² |
| Patient (ID #) | of BSA) | of BSA) | of BSA) |

| | | | | |
|---|---|---|---|---|
| 1 | (KKD-001) | 286.7273 | 318.9275 | 306.2139 |
| 2 | (JSS-002) | 82.04539 | 132.7069 | 151.0555 |
| 3 | (STL-003) | 152.5714 | 161.955 | 145.7199 |
| 4 | (JHU-001) | 159.6719 | | 134.5482 |
| 5 | (501-ABG-001) | 169.012 | 113.4526 | 112.1804 |
| 6 | (501-DTT-003) | 176.5842 | 137.0677 | 116.0211 |
| 7 | (504-LXW-001) | 60.24383 | 136.0326 | 139.4345 |
| 8 | (501-AJS-004) | 164.3465 | 173.3984 | 197.4093 |
| 9 | (504-OXF-002) | 128.9999 | 194.71 | |
| 10 | (506-ILB-001) | 138.0816 | 109.8419 | 127.138 |
| 11 | (506-S-S-002) | 124.2315 | 167.8823 | 118.0871 |
| 12 | (501-A-S-005) | 159.7195 | 194.9616 | |
| 13 | (501-M-D-007) | 115.564 | 141.2428 | 157.8121 |
| 14 | (506-JSB-003) | 99.06859 | | 99.44577 |

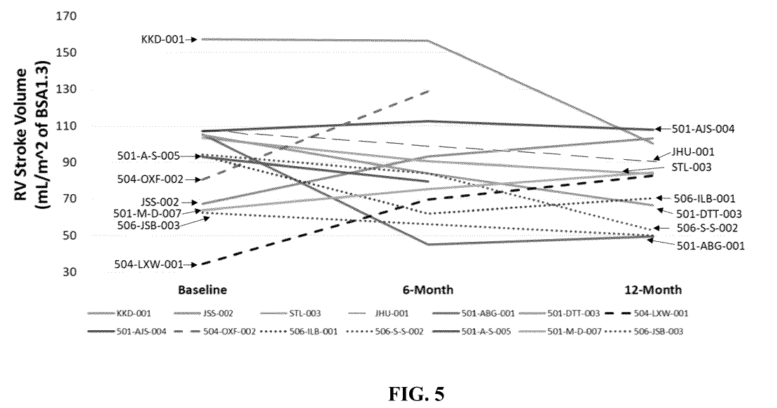

FIG. 5 depicts the stroke volume changes for each patient throughout the course of the clinical study. Measurements were taken at the beginning of the clinical study, six months post administration and twelve months post administration. The data presented within FIG. 5 was indexed to the BSA of the patients. Table 8 contains the MRI data used to determine the change in each patient's stroke volume after administration of the Lomecel-B™ cells.

TABLE 8
Change in the Stroke Volume of Each Patient after
Lomecel-B ™ Administration

| | | | RV SV | RV SV |
|---|---|---|---|---|
| | | RV SV Baseline | 6-Month | 12-Month |
| | | (mL/m² | (mL/m² | (mL/m² |
| Patient (ID #) | | of BSA) | of BSA) | of BSA) |
| 1 | (KKD-001) | 157.2375 | 156.455 | 100.2647 |
| 2 | (JSS-002) | 67.12805 | 93.17722 | 103.1013 |
| 3 | (STL-003) | 103.3548 | 90.39349 | 83.71142 |
| 4 | (JHU-001) | 107.7785 | | 90.67376 |
| 5 | (501-ABG-001) | 105.0615 | 44.78393 | 49.24993 |
| 6 | (501-DTT-003) | 104.996 | 83.91901 | 66.29779 |
| 7 | (504-LXW-001) | 34.42505 | 69.46344 | 82.62783 |
| 8 | (501-AJS-004) | 107.0163 | 112.3879 | 107.6778 |
| 9 | (504-QXF-002) | 80.62496 | 128.6477 | |
| 10 | (506-ILB-001) | 93.40812 | 61.96209 | 70.37999 |
| 11 | (506-S-S-002) | 94.24457 | 83.94114 | 52.76234 |
| 12 | (501-A-S-005) | 92.8602 | 79.52381 | |
| 13 | (501-M-D-007) | 63.75947 | 75.3295 | 84.54217 |
| 14 | (506-JSB-003) | 62.37652 | | 49.72289 |

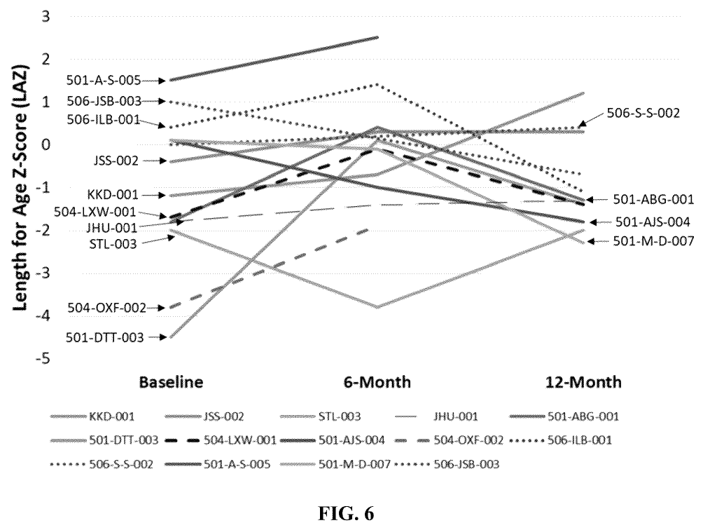

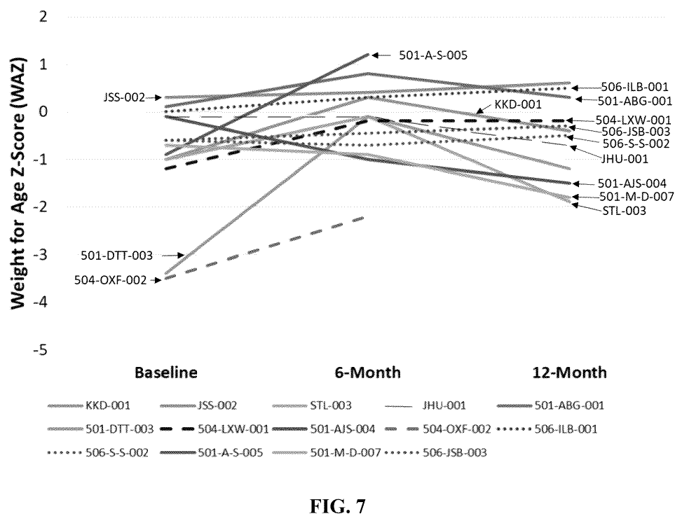

In addition to examining the volume and mass changes in the right ventricular, somatic growth was also examined for every patient. The somatic growth of each patient was measured in terms of age- and length/weight-adjusted Z-scores, which is the standard deviation above or below the mean of the general population. A Z-score of 0 is equivalent of 50th percentile, with positive addition going to higher percentiles and vice versa. FIG. 6 depicts the change in the length-for-age Z-scores of each patient at the beginning of the clinical study, six months post administration and twelve months post administration. FIG. 7 depicts the change in the weight-for-age Z-scores of each patient at the beginning of the clinical study, six months post administration and twelve months post administration. Table 9 contains the data used to determine the change in each patient's length-for-age Z-scores after administration of the Lomecel-B™ cells. Table 10 contains the data used to determine the change in each patient's weight-for-age Z-scores after administration of the Lomecel-B™ cells.

TABLE 9
Change in Somatic Growth (Length-for-Age Z-scores) of Each
Patient after Lomecel-B ™ Administration

| | | | LAZ | LAZ |
|---|---|---|---|---|
| Patient (ID #) | | LAZ Baseline | 6-Month | 12-Month |
| 1 | (KKD-001) | −1.2 | −0.7 | 1.2 |
| 2 | (JSS-002) | −0.4 | 0.3 | 0.3 |
| 3 | (STL-003) | −2 | −3.8 | −2 |
| 4 | (JHU-001) | −1.8 | −1.4 | −1.3 |
| 5 | (501-ABG-001) | −1.8 | 0.4 | −1.3 |
| 6 | (501-DTT-003) | −4.5 | 0.1 | −1.4 |
| 7 | (504-LXW-001) | −1.7 | −0.1 | −1.4 |
| 8 | (501-AJS-004) | 0.1 | −1 | −1.8 |
| 9 | (504-OXF-002) | −3.8 | −1.9 | |
| 10 | (506-ILB-001) | 0.4 | 1.4 | −1.1 |
| 11 | (506-S-S-002) | 0 | 0.2 | 0.4 |
| 12 | (501-A-S-005) | 1.5 | 2.5 | |
| 13 | (501-M-D-007) | 0.1 | −0.1 | −2.3 |
| 14 | (506-JSB-003) | 1 | | −0.7 |

TABLE 10
Change in Somatic Growth (Weight-for-Age Z-scores) of Each
Patient after Lomecel-B ™ Administration

| | | | WAZ | WAZ |
|---|---|---|---|---|
| Patient (ID #) | | WAZ Baseline | 6-Month | 12-Month |
| 1 | (KKD-001) | −1 | 0.3 | −0.4 |
| 2 | (JSS-002) | 0.3 | 0.4 | 0.6 |
| 3 | (STL-003) | −1 | −0.1 | −1.9 |
| 4 | (JHU-001) | −0.1 | −0.1 | −0.7 |
| 5 | (501-ABG-001) | 0.1 | 0.8 | 0.3 |
| 6 | (501-DTT-003) | −3.4 | −0.1 | −1.2 |
| 7 | (504-LXW-001) | −1.2 | −0.2 | −0.2 |
| 8 | (501-AJS-004) | −0.1 | −1 | −1.5 |
| 9 | (504-OXF-002) | −3.5 | −2.2 | |
| 10 | (506-ILB-001) | 0 | 0.3 | 0.5 |
| 11 | (506-S-S-002) | −0.6 | −0.7 | −0.5 |
| 12 | (501-A-S-005) | −0.9 | 1.2 | |
| 13 | (501-M-D-007) | −0.7 | −0.9 | −1.8 |
| 14 | (506-JSB-003) | −0.6 | | −0.3 |

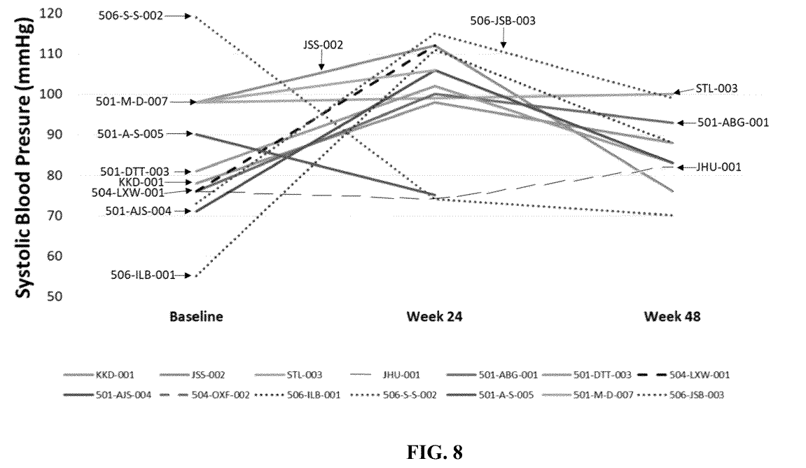

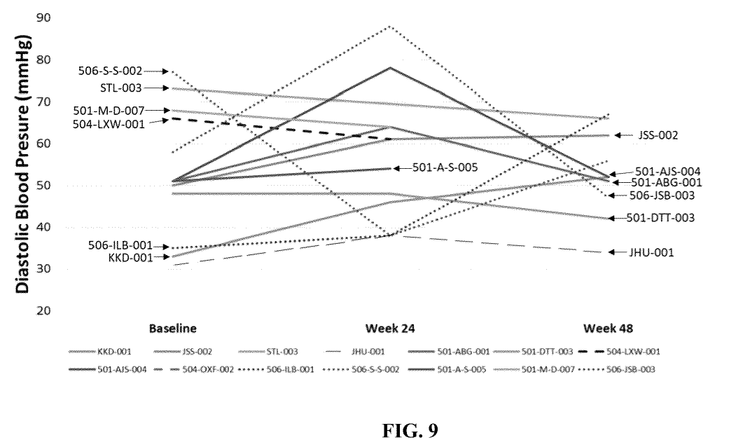

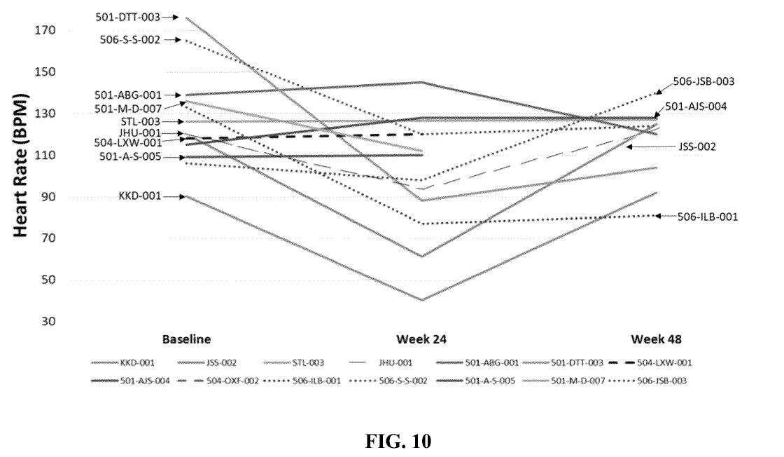

The blood pressure and heart rate of each patient was also examined during the clinical study. Both blood pressure and heart rate were measured for each patient at the beginning of the clinical study, 24 weeks post administration and 48 weeks post administration. FIG. 8 depicts the change in systolic blood pressure for each patient after administration. FIG. 9 depicts the change in diastolic blood pressure for each patient after administration. FIG. 10 depicts the change in heart rate for each patient after administration. Table 11 contains the data used to determine the change in each patient's systolic blood pressure after administration of the Lomecel-B™ cells. Table 12 contains the data used to determine the change in each patient's diastolic blood pressure after administration of the Lomecel-B™ cells. Table 13 contains the data used to determine the change in each patient's heart rate after administration of the Lomecel-B™ cells.

TABLE 11
Change in Systolic Blood Pressure of Each Patient
after Lomecel-B ™ Administration

| | | | SBP | SBP |
|---|---|---|---|---|
| | | SBP Baseline | 6-Month | 12-Month |
| Patient (ID #) | | (mmHg) | (mmHg) | (mmHg) |
| 1 | (KKD-001) | 78 | 98 | 88 |
| 2 | (JSS-002) | 98 | 112 | 76 |
| 3 | (STL-003) | 98 | | 100 |
| 4 | (JHU-001) | 76 | 74 | 82 |
| 5 | (501-ABG-001) | 76 | 100 | 93 |
| 6 | (501-DTT-003) | 81 | 102 | 83 |
| 7 | (504-LXW-001) | 76 | 112 | |
| 8 | (501-AJS-004) | 71 | 106 | 83 |
| 9 | (504-OXF-002) | 71 | | |
| 10 | (506-ILB-001) | 55 | 111 | 88 |
| 11 | (506-S-S-002) | 119 | 74 | 70 |
| 12 | (501-A-S-005) | 90 | 75 | |
| 13 | (501-M-D-007) | 98 | 106 | |
| 14 | (506-JSB-003) | 73 | 115 | 99 |

TABLE 12
Change in Diastolic Blood Pressure of Each Patient
after Lomecel-B ™ Administration

| | | | DBP | DBP |
|---|---|---|---|---|
| | | DBP Baseline | 6-Month | 12-Month |
| Patient (ID #) | | (mmHg) | (mmHg) | (mmHg) |
| 1 | (KKD-001) | 33 | 46 | 52 |
| 2 | (JSS-002) | 50 | 61 | 62 |
| 3 | (STL-003) | 73 | | 66 |
| 4 | (JHU-001) | 31 | 38 | 34 |
| 5 | (501-ABG-001) | 51 | 64 | 51 |
| 6 | (501-DTT-003) | 48 | 48 | 42 |
| 7 | (504-LXW-001) | 66 | 61 | |
| 8 | (501-AJS-004) | 51 | 78 | 52 |
| 9 | (504-OXF-002) | 55 | | |
| 10 | (506-ILB-001) | 35 | 38 | 67 |
| 11 | (506-S-S-002) | 77 | 38 | 56 |
| 12 | (501-A-S-005) | 51 | 54 | |
| 13 | (501-M-D-007) | 68 | 64 | |
| 14 | (506-JSB-003) | 58 | 88 | 47 |

TABLE 13
Change in Heart Rate of Each Patient after
Lomecel-B ™ Administration

| | | HR | HR |
|---|---|---|---|
| | HR Baseline | 6-Month | 12-Month |
| Patient (ID #) | (BPM) | (BPM) | (BPM) |

| | | | | |
|---|---|---|---|---|
| 1 | (KKD-001) | 90 | 40 | 92 |
| 2 | (JSS-002) | 120 | 61 | 125 |
| 3 | (STL-003) | 126 | | 127 |
| 4 | (JHU-001) | 120 | 94 | 122 |
| 5 | (501-ABG-001) | 139 | 145 | 120 |
| 6 | (501-DTT-003) | 176 | 88 | 104 |
| 7 | (504-LXW-001) | 118 | 120 | |
| 8 | (501-AJS-004) | 115 | 128 | 128 |
| 9 | (504-OXF-002) | 119 | | |
| 10 | (506-ILB-001) | 133 | 77 | 81 |
| 11 | (506-S-S-002) | 165 | 120 | 124 |
| 12 | (501-A-S-005) | 109 | 110 | |
| 13 | (501-M-D-007) | 136 | 112 | |
| 14 | (506-JSB-003) | 106 | 98 | 140 |

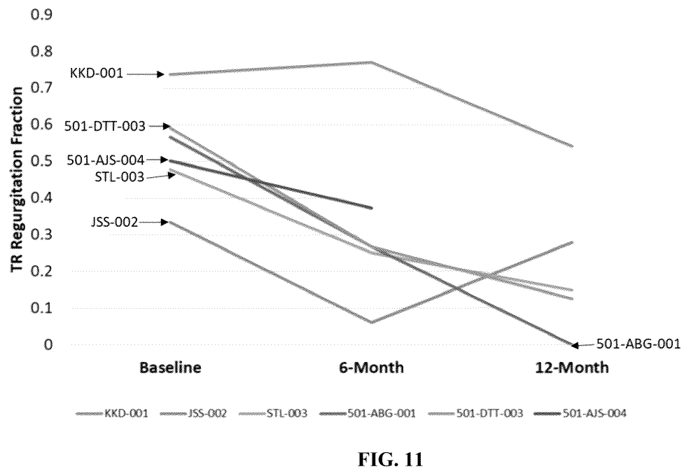

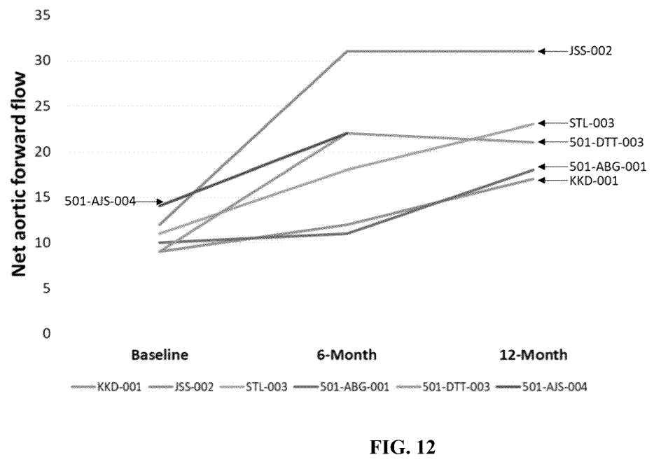

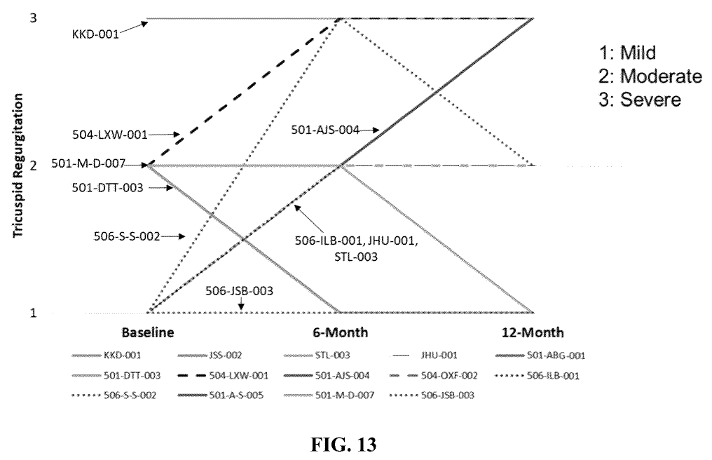

The tricuspid regurgitation of each patient was also examined during the clinical study. FIG. 11 depicts the change in tricuspid regurgitation fraction for select patients at the beginning of the clinical study, six months post administration and twelve months post administration. FIG. 12 depicts the change in tricuspid regurgitation net aortic forward flow for select patients at the beginning of the clinical study, six months post administration and twelve months post administration. FIG. 13 depicts the change in each patient's tricuspid regurgitation at the beginning of the clinical study, six months post administration and twelve months post administration. Table 14 contains the data used to determine the change in each select patient's tricuspid regurgitation fraction after administration of the Lomecel-B™ cells. Table 15 contains the data used to determine the change in each select patient's tricuspid regurgitation net aortic forward flow after administration of the Lomecel-B™ cells. Table 16 contains the data used to determine the change in each patient's tricuspid regurgitation after administration of the Lomecel-B™ cells.

TABLE 14
Change in Tricuspid Regurgitation Fraction of Select Patients
after Lomecel-B ™ Administration

| | | TR RF | TR RF |
|---|---|---|---|
| Patient (ID #) | TR RF Baseline | 6-Month | 12-Month |
| KKD-001 | 0.735294 | 0.769231 | 0.540541 |
| JSS-002 | 0.333333 | 0.060606 | 0.27907 |
| STL-003 | 0.47619 | 0.25 | 0.148148 |
| 501-ABG-001 | 0.565217 | 0.266667 | 0 |
| 501-DTT-003 | 0.590909 | 0.266667 | 0.125 |
| 501-AJS-004 | 0.5 | 0.371429 | |

TABLE 15
Change in Tricuspid Regurgitation Net Aortic Forward Flow of Select
Patients after Lomecel-B ™ Administration

| | TR NAFF | TR NAFF | TR NAFF |
|---|---|---|---|
| Patient (ID #) | Baseline | 6-Month | 12-Month |
| KKD-001 | 9 | 12 | 17 |
| JSS-002 | 12 | 31 | 31 |
| STL-003 | 11 | 18 | 23 |
| 501-ABG-001 | 10 | 11 | 18 |
| 501-DTT-003 | 9 | 22 | 21 |
| 501-AJS-004 | 14 | 22 | |

TABLE 16
Change in Tricuspid Regurgitation of Each Patient
after Lomecel-B ™ Administration

| | | | |
|---|---|---|---|
| | | TR | TR |
| Patient (ID #) | TR | 6-Month | 12-Month |

| | | | | |
|---|---|---|---|---|
| 1 | (KKD-001) | 3 | 3 | 3 |
| 2 | (JSS-002) | 1 | 2 | 2 |
| 3 | (STL-003) | 1 | 2 | 2 |
| 4 | (JHU-001) | 1 | 2 | 2 |
| 5 | (501-ABG-001) | 2 | 1 | 1 |
| 6 | (501-DTT-003) | 2 | 1 | 1 |
| 7 | (504-LXW-001) | 2 | 3 | 3 |
| 8 | (501-AJS-004) | 2 | 2 | 3 |
| 9 | (504-OXF-002) | 2 | 2 | |
| 10 | (506-ILB-001) | 1 | 2 | 1 |
| 11 | (506-S-S-002) | 1 | 3 | 2 |
| 12 | (501-A-S-005) | 2 | 2 | |
| 13 | (501-M-D-007) | 2 | 2 | 1 |
| 14 | (506-JSB-003) | 1 | | 1 |

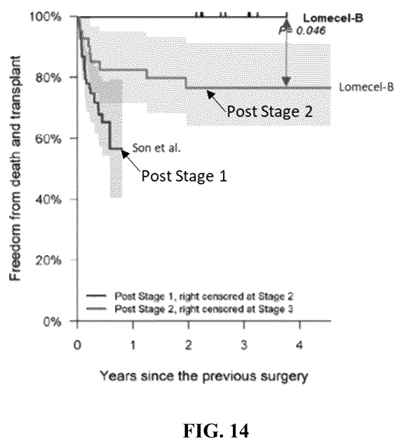

The average post-administration survival rate for each patient was also measured and compared against the survival rate of patients enrolled in previous HLHS clinical studies, specifically the clinical study performed by Son et al. (Son et al., “Prognostic value of serial echocardiography in hypoplastic left heart syndrome”. Circulation: Cardiovascular Imaging, (2018) 11 (7), e006983). FIG. 14 depicts this comparison.

### Study Findings

Intramyocardial injection of Lomecel-B™ product was well-tolerated, with no MACE, and no infections or any other adverse events reported that were considered to be related to investigational treatment. The efficacy results from this trial involved improvement in patient survival and perseverance of the RV function.

In summary, treatment with Lomecel-B™ in HLHS patients was safe and showed encouraging clinical outcomes, indicating higher transplant-free survival than Stage II surgery without Lomecel-B™ (historical control) and preservation of RV contractility as measured by GLS. These clinical findings demonstrate the potential for Lomecel-B™ product to treat HLHS and reduce mortality and the need for heart transplant.

We claim:

1. A method for treating juvenile hypoplastic left heart syndrome in a patient in need thereof, the method comprising administering a therapeutically effective amount of allogenic mesenchymal stem cells to the patient in need thereof.

2. The method of claim 1, wherein the therapeutically effective amount is from about 20×10⁶ to about 100×10⁶ allogenic mesenchymal stem cells.

3. The method of claim 1, further comprising measuring a change in the patient's right ventricular mass after administration of the therapeutically effective amount of allogenic mesenchymal stem cells.

4. The method of claim 3, wherein the change in the patient's right ventricular mass after administration is an increase in right ventricular mass from about 0.1% to about 10%.

5. The method of claim 1, further comprising measuring a change in the patient's right ventricular ejection fraction after administration of the therapeutically effective amount of allogenic mesenchymal stem cells.

6. The method of claim 5, wherein the change in the patient's right ventricular ejection fraction after administration is a decrease in right ventricular ejection fraction from about 0.1% to about 10%.

7. The method of claim 1, further comprising measuring a change in the patient's right ventricular end-systolic volume after administration of the therapeutically effective amount of allogenic mesenchymal stem cells.

8. The method of claim 7, wherein the change in the patient's right ventricular end-systolic volume after administration is an increase in right ventricular end-systolic volume from about 0.1% to about 10%.

9. The method of claim 1, further comprising measuring a change in the patient's right ventricular end-diastolic volume after administration of the therapeutically effective amount of allogenic mesenchymal stem cells.

10. The method of claim 9, wherein the change in the patient's right ventricular end-diastolic volume after administration is an increase in right ventricular end-diastolic volume from about 0.1% to about 10%.

11. The method of claim 1, further comprising measuring a change in the patient's stroke volume after administration of the therapeutically effective amount of allogenic mesenchymal stem cells.

12. The method of claim 1, further comprising measuring a change in the patient's length-for-age Z-scores after administration of the therapeutically effective amount of allogenic mesenchymal stem cells.

13. The method of claim 1, further comprising measuring a change in the patient's weight-for-age Z-scores after administration of the therapeutically effective amount of allogenic mesenchymal stem cells.

14. The method of claim 1, further comprising measuring a change in the patient's systolic blood pressure after administration of the therapeutically effective amount of allogenic mesenchymal stem cells.

15. The method of claim 1, further comprising measuring a change in the patient's diastolic blood pressure after administration of the therapeutically effective amount of allogenic mesenchymal stem cells.

16. The method of claim 1, further comprising measuring a change in the patient's heart rate after administration of the therapeutically effective amount of allogenic mesenchymal stem cells.

17. The method of claim 1, wherein the therapeutically effective amount of allogenic mesenchymal stem cells is administered to the patient in need thereof by intramyocardial injection.

18. The method of claim 1, wherein the therapeutically effective amount of allogenic mesenchymal stem cells is administered to the patient in need thereof as a single dose.

19. The method of claim 1, wherein the patient in need thereof is from 1 to 15 years old.

20. The method of claim 1, wherein the allogeneic human mesenchymal stem cells are derived from bone marrow and/or adipose tissue.

21. The method of claim 1, further comprising measuring a change in the patient's tricuspid regurgitation fraction after administration of the therapeutically effective amount of allogenic mesenchymal stem cells.

22. The method of claim 1, further comprising measuring a change in the patient's tricuspid regurgitation net aortic forward flow after administration of the therapeutically effective amount of allogenic mesenchymal stem cells.

23. The method of claim 1, further comprising measuring the patient's survival rate after administration of the therapeutically effective amount of allogenic mesenchymal stem cells.
